(19) United States
(12) Patent Application Publication
(10) Pub. No.: US 2024/0423910 A1
(43) Pub. Date: Dec. 26, 2024

(54) METHOD AND APPARATUS FOR MAKING A TREATMENT SOLUTION AND FOR PROVIDING SAID TREATMENT SOLUTION TO A BIOLOGICAL SYSTEM

(71) Applicant: SWISS.318 SAGL, Breganzona, Lugano (CH)

(72) Inventor: Roberto ALBERTELLI, Forte dei Marmi (LU) (IT)

(73) Assignee: SWISS.318 SAGL, Breganzona, Lugano (CH)

(21) Appl. No.: 18/698,026

(22) PCT Filed: Oct. 7, 2022
(86) PCT No.: PCT/IB2022/059641

§ 371 Date: Apr. 3, 2024

(30) Foreign Application Priority Data
Oct. 7, 2021 (IT) 102021000025622

(51) Int. Cl. A61K 9/08; A61K 33/00; A61K 33/40; A61K 47/02; B01F 23/231; B01F 23/232; B01F 25/40; B01J 13/00; C01B 13/10

(52) U.S. Cl. CPC A61K 9/08; A61K 33/00; A61K 33/40; A61K 47/02; B01F 23/2312; B01F 23/232; B01F 25/40; B01J 13/0052; C01B 13/10

(57) ABSTRACT

An apparatus and method for obtaining a treatment solution containing reactive oxygen species includes generating an ozone water solution and feeding it into a first of two apertures of a container at a predetermined pressure. The container including a passage body comprising a plurality of passageways having a predetermined passage-section size, so that the ozone water solution flows through the passage body generating the treatment solution in which the ozone is converted to reactive oxygen, and so that the treatment solution then flows through a nebulizer device and is released in the form of small droplets immediately after it has been generated, in which, the reactive oxygen species interact with the biological system before they can decay, or can incorporated into a gel. The traversing body may be a bundle of fibers, a porous body with a percolable open-cell lattice, or a bed of a loose mineral particulate material.

### SCOPE OF THE INVENTION

The present invention relates to a method for making a water solution containing reactive oxygen species (ROS) and for delivering the treatment solution with negligible or acceptable decay of the reactive oxygen species. The invention also relates to an apparatus for making such a treatment solution and for delivering the treatment solution to a biological system with negligible or acceptable decay of the reactive oxygen species.

### BRIEF DESCRIPTION OF THE PRIOR ART

Reactive oxygen species (ROS) are oxygen compounds that have one or more unpaired electrons, and are therefore strongly reactive. Examples thereof include superoxide anion O₂⁻, hydroxyl radical OH and so forth.

As well known, reactive oxygen species are involved in various cellular processes, in particular they accelerate plant and animal cell metabolism as described, for instance, in Italian patent application No. 102018000009939.

IT 102018000009939 describes an apparatus for obtaining a solution of ozone splitting products, in particular superoxide anions O₂⁻. The apparatus includes a tubular ozone generator for obtaining an ozone-containing gas from air or oxygen; a helical-tube ion generator, in which the ozone-containing gas flows through a strong alternating electric field; a magnetic ion separator where superoxide anions O₂⁻ present in the gas are separated from singlet oxygen O¹₂; a heat exchanger to cool down the gas leaving the magnetic separator; and a micronizer to absorb this gas into water in the form of microbubbles.

The above equipment is complicated and expensive, includes such energy-intensive components as the ion generator and the magnetic separator, and also requires vigorous cooling of the superoxide anions-containing stream. Moreover, in the obtained solution or dispersion, the superoxide anions are present at a low concentration, and rapidly decay to molecular oxygen.

EP3804844A1 discloses a generator of nanobubbles in water. A gas, that can be also ozone, is supplied along with a water supply to a ultrafine bubble generating apparatus, which is capable of generating water containing nanobubbles, which in turn is added to a water main stream at a junction point. The water main stream and the water containing nanobubbles then are sprayed on an acceptor like a plant. In this way, the water containing nanobubbles of the gas is sprayed on the acceptor. If ozone is used as gas, then on the acceptor a water containing nanobubbles of ozone is prayed on the acceptor. Passage holes are provided in bubble generating apparatus for generating the nanobubbles.

CN111643700A discloses a movable ready-to-use ozone disinfection sprayer, comprising an ozone-water synthesis device and an ozone-water dissolving system. The ozone generated by generator is dissolved in water through the ozone water dissolving system, obtaining a high-concentration ozone water capable of killing bacteria and virus. Since the ozone-water synthesis device only requires water, the movable ready-to-use ozone disinfection sprayer only requires adding water into a tank. The ozone-water dissolving system comprises a water-ozone dissolution tube where ozone and water mix before being sprayed by a nozzle.

JPH0240289A discloses an ozonizer which generates ozone that is fed into water. By using such water a shelf stability of perishables is increased and life of fishes in such water is increased. A passage of a water pipe 26 receives ozone from a nozzle supplied through a tube. In order to cause mixing of the ozone into water, a gas-liquid mixing part is provided downstream of the nozzle, comprising a meshy body containing plural porous members of ceramic. Bubbles of ozone in water flowing out of a passage are dissolved in water by passing through members.

DE2938517A1 discloses a device for enriching water with O₃ to produce a sterilizing solution for medical and dental practice. The line has a mixing nozzle to which gaseous O₃ is injected. The nozzle has a mesh insert which serves to mix the water and gas. In particular, a head is connected to the front end of a mixing nozzle containing a housing having an inlet for receiving flowing water (arrow) and a further inlet to which a line can be connected, through which ozone can flow (arrow), coming from an ozonizing device or from an ozone store. Between the inlet for the ozone and an outlet of mixing nozzle a plurality of individual screens that are stacked next to one another and which serve as a multistage mixing screen for intimately mixing ozone and water and thus for producing an ozone/water mixture which is enriched in high concentration. Plates and fine-mesh grids can be provided. The mesh divides the flow and provides an intimate mixture between the gas and the liquid. The device can be incorporated into a dentist's drill.

### SUMMARY OF THE INVENTION

It is therefore an object of the present invention to provide a method and an apparatus for making a ROS-containing product, in particular a superoxide anions-containing product, in which the latter can be delivered before an unacceptable amount of the ROS decays back to molecular oxygen.

It is a particular object of the invention to provide such a method and apparatus in which the ROS-containing product is a water solution that can be supplied to the biological system immediately after the ROS production.

It is also an object of the invention to provide such a method and apparatus to achieve a higher superoxide anions concentration in the product or solution.

It is also an object of the invention to provide such a method and apparatus to obtain the above product with a lower energy consumption than in the prior art.

It is another object of the invention to provide such an apparatus that is simpler and less expensive to manufacture than the prior art devices.

It is also an object of the invention to provide such an apparatus that is small in size and can be easily transported.

The above-mentioned purposes are achieved by a method and an apparatus for producing a treatment solution containing reactive oxygen species capable of treating a biological system from an ozone water solution, as defined in claim 1, and by an apparatus for making such a treatment solution and for treating a biological system with the aqueous treatment solution, as defined in claim 12. Advantageous variants and embodiments of the method and apparatus are defined in the dependent claims.

For the sake of conciseness, the term ‘ozone water solution’ will be used in the following description to refer to the ozone water solution, and the term ‘treatment solution’ to refer to the aqueous treatment solution containing reactive oxygen species.

According to one aspect of the invention, the method comprises the steps of:
prearranging a container with a first opening and a second opening and a passage body within the container, between the first opening and the second opening,
wherein the passage body includes a plurality of passageways having a predetermined passage-section size;
prearranging a nebulizer device in hydraulic connection with the second opening;
generating an ozone water solution;
feeding the ozone water solution into the first opening of the container at a predetermined first pressure, in such a way that
   the ozone water solution is caused to flow through the plurality of passageways of the passage body, wherein said predetermined passage-section size is selected to convert the ozone in the ozone water solution is converted to reactive oxygen species, generating a treatment solution at a second pressure lower than the first pressure; the treatment solution is immediately thereafter caused to flow through the nebulizer device and is then nebulized into submillimetric particles which can be directly deposited on the biological system and can interact with it without decaying into molecular oxygen O₂ or which can be preserved,
wherein the passage body is configured to cause said pressure to drop from the first pressure value of the ozone water solution to the second pressure value of the treatment solution passing therethrough, and is selected from the group consisting of:
   a bundle of hollow fibres, and the passageways for the ozone water solution and for the treatment solution are defined within the hollow fibres;
   a bundle of full fibres, and the passageways, through which the ozone water solution turning into the treatment solution flows, include empty spaces among the full fibres;
   a porous body, wherein the passageways, through which the ozone water solution turning into the treatment solution flows, are defined by a percolable open-cell lattice having a predetermined cell size,
   a body (76′) comprising a loose mineral particulate material having a particle size selected, also in this case, in such a way that a leachable open-cell lattice is formed having a predetermined cell size;
   a combination of the above-mentioned passage bodies.

According to another aspect of the invention, the apparatus comprises:
an ozone water solution generator device;
a treatment solution generator device for generating a ROS water solution including, in particular superoxide anions,
wherein the treatment solution generator device is arranged in hydraulic connection with the ozone water solution generator device,
wherein the treatment solution generator device comprises:
   a container with a first opening and a second opening, and
   a passage body arranged within the container between the first opening and the second opening of the container,
wherein the passage body includes a plurality of passageways having a predetermined passage-section size;
   a nebulizer device in hydraulic connection with the second container opening of the container, the nebulizer device arranged to release the treatment solution outside of the treatment solution generating device in the form of submillimetric particles;
a supply pump arranged to supply the ozone water solution into the first opening of the container at a predetermined first pressure, in such a way that
   said ozone water solution flows through the passage body, forming the treatment solution at a second pressure lower than the first pressure;
   said treatment solution at the second pressure subsequently flows through the nebulizer and is released outside of the treatment solution generator device,
   wherein the plurality of passageways of the passage body, present the predetermined passage-section size selected to convert the ozone of the ozone water solution into the treatment solution, in such a way that reactive oxygen species are dissolved in the treatment solution and can be directly sprinkled by the nebulizer device without decaying into molecular oxygen O₂,
wherein the passage body is configured to cause said pressure to drop from the first pressure value of the ozone water solution to the second pressure value of the treatment solution passing therethrough, and is selected from the group consisting of:
   a bundle of hollow fibres, and the passageways for the ozone water solution and for the treatment solution are defined within the hollow fibres;
   a bundle of full fibres, and the passageways are defined by empty spaces among the full fibres;
   a porous body, wherein the passageways are defined by a percolable open-cell lattice having a predetermined cell size;
   a combination of the above-mentioned passage bodies.

The pressurised flow of the ozone water solution through passageways with a narrow passage dimension assists the conversion of the intrinsically unstable O₃ molecules into reactive oxygen species. In fact, the combined action of the pressure, set between the first pressure and the second pressure, and the narrow passage section of the passage body passageways, through which the water solution flows, induces a mechanical stress on the O₃ molecules that leads to the O₃-splitting towards the aforementioned ionic species O₂⁻ and O⁺, i.e. to the heterolytic cleavage reaction:

2O₃→O₂⁻+O⁺.

The “biological system” can include plants, seeds, human cells and animal cells. By administrating the treatment solution to such a biological system directly or immediately, or in a gel-stabilized form, at the ROS concentrations allowed by the production method and apparatus, and/or by possible ROS gel-stabilization, enables reactive oxygen species to accelerate cellular metabolic processes. This results in beneficial effects such as, for instance, accelerated growth of plants, increased size of plants themselves and of their fruits, quick human or animal tissue regeneration, in particular of skin tissue, hair bulbs reactivation and other effects the inventor has found out.

In particular, the bundle of hollow fibres may be made of a material selected among a polysulphone, cellulose triacetate and polyvinyl chloride.

In particular, the porous body may comprise a sintered, ceramic or metallic material, or an open cell sponge.

For instance, the loose mineral particulate material may comprise an appropriately screened sand or an ultrafiltration aid.

The above-mentioned embodiments of the passage body are all simple and relatively cheap to manufacture, and therefore they allow containing the manufacturing costs of the apparatus.

Advantageously, the passage-section size of the passageways in the passage body is set in a range appropriate to obtain an ultrafiltration step of a fluid flowing therethrough. For instance, the walls of the hollow fibres can have a submicrometric porosity, for example they can be hollow fibres of one of the types used in haemodialysis. In this case, advantageously, the porosities of the wall of the hollow fibres can be configured as a part of the passageways through which the water solution containing ozone and the ROS being formed flows.

In particular, the passage-section size of the passageways is set between 0.001 μm and 0.2 μm, preferably between 0.005 μm and 0.015 μm. The aforementioned size ranges make it possible to increase, or maximize the O₂⁻ and O⁺ concentrations in the treatment solution. The forced flow of the ozone water solution through the passageways determines the pressure drop from the first pressure to the second pressure.

The supply pump is selected in such a way that the ozone water solution reaches the container of treatment solution generator device at a predetermined first pressure P₁, preferably set between 7 and 15 bar g, in particular between 10 and 12 bar g. Moreover, the passage body is arranged to release the freshly formed treatment solution to nebulizer device at second pressure P₂ set between 4 bar g and 7 bar g.

Advantageously, before or after passing through the passageways the ozone water solution, or the ROS-containing treatment solution, respectively, can flow through duct portions that are provided in the passage body. Said duct portions can have a section size set between 10 μm and 1 mm, preferably between 50 μm and 500 μm, more preferably between 100 μm and 300 μm. The above duct portios make easier the flow of the water solutions.

In particular, the step of generating an ozone water solution comprises the steps of:
generating ozone from oxygen, in particular from oxygen of an air stream, obtaining an ozone-containing gas stream;
contacting the ozone-containing gas stream with water in a mixer device and dissolving the ozone into the water, so as to form the ozone water solution.

To this purpose, the ozone water solution generator device may include:
a water-ozone mixer device;
an ozone generator pneumatically connected to the mixer device and arranged to receive an atmospheric air stream and to change the latter into a stream of ozone-containing gas;
an ozone supply unit for supplying said ozone to the mixer device, the supply unit arranged to bring the ozone-containing gas stream into contact with water contained in the mixer device, so that the ozone dissolves into the water, forming an ozone water solution.

In one embodiment, the mixer device comprises a reservoir configured to contain a predetermined amount of water. In this case, the ozone supply unit and reservoir are mutually arranged to supply the stream of ozone-containing gas into the reservoir below a level corresponding to the amount of water therein. In this embodiment, the ozone water solution is generated batchwise in the reservoir. which allows for more accurate control of the ozone concentration in the ozone water solution and the concentration of the reactive oxygen species in the treatment solution can be more accurately controlled as well.

In another embodiment, the mixer device comprises a mixing duct within which a static mixer is arranged. This way, the ozone water solution is formed while flowing through the mixing duct, in a continuous way like the subsequent step of generating the treatment solution. Therefore, no storage container for the ozone water solution is required, so the size and the weight of the apparatus can be contained. For this reason, the transport and the use of the apparatus are simplified. Moreover, in this embodiment, the ozone water solution is used to generate the treatment solution immediately after being produced and the ozone of the water solution is immediately converted into ROS. Therefore, only a small amount of ozone is turned back to diatomic oxygen O₂, due to its instability, before being converted into ROS. This increases the overall ROS yield in the ROS generation process from oxygen.

Advantageously, the water used to dissolve the ozone and to form the ozone water solution includes a predetermined amount of hydrogen peroxide, therefore the step of contacting and dissolving the ozone into the water takes place in the presence of hydrogen peroxide.

Preferably, the hydrogen peroxide amount is equivalent to a 35% hydrogen peroxide water solution amount set between 1/500 and 1/5000 of the amount of said water.

Preferably, the water used to make the ozone water solution contains a certain amount of dissolved salts. Specifically, the dissolved salts consist of at least one pair of an anion and one cation selected from the group consisting of Sodium, Potassium, Calcium, Magnesium, Iron, Chloride, Sulphate, Bicarbonate, Fluoride, Nitrate, each present at a concentration set between 50 and 500 mg/litre.

Preferably, the container of the treatment solution generating device has an elongated shape and the first and second openings are arranged at opposite end portions of the container. In particular, the passage body or the container has a tubular shape of length set between 3 and 5 times the diameter.

It falls within the scope of the invention also a method for stabilizing reactive oxygen species, including superoxide anion O₂⁻, in a treatment solution manufactured as described above, by incorporation of the submillimetric particles of the treatment solution into a gel.

In particular, such incorporation of ROS water solution submillimetric particles into a gel includes the steps of:
prearranging an amount of a gel in a gel-forming turbomixer, i.e., in an equipment commonly used to make a gel;
maintaining the gel under agitation in the gel-forming turbomixer;
supplying the treatment solution, as released by the nebulizer device, into the gel-forming turbomixer during the above step of agitation of the gel.

In particular, the gel can be produced in situ, i.e. in the gel-forming turbomixer just before or even during the incorporation of the ROS water solution in the gel formed or being formed, by prearranging an amount of a gelling agent in the gel-forming turbomixer and by supplying a predetermined amount of water (8) to said gel-forming turbomixer (90), during the above step of agitation of the gel

To this purpose, the apparatus comprises such a gel-forming turbomixer hydraulically connected to an output mouth of the nebulizer device so as to be supplied by the treatment solution as released thereby. Moreover, the gel-forming turbomixer can be associated with a gelling agent supply means and by a water supply means so as to prepare said gel within said gel-forming turbomixer, or to compensate for the viscosity change of a preformed gel due to the addition of water accompanying the ROS in the treatment solution.

### BRIEF DESCRIPTION OF THE DRAWINGS

Further features and/or advantages of the present invention will become clearer with the following description of variants and forms of embodiment, made by way of example and not limitation, with reference to the accompanying drawings in which

FIG. 1 is a flow diagram of an apparatus for making a treatment solution containing reactive oxygen species according to the present invention;

FIG. 2 diagrammatically shows a longitudinal cross section view of a treatment solution generator device in which the passage body is made of full fibres;

FIG. 3 diagrammatically shows a longitudinal cross section view of a treatment solution generator device in which the passage body is made of hollow fibres;

FIG. 4 is a detail of the fibres of passage body of FIG. 4, in a modification in which microporous fibres are used;

FIG. 5 diagrammatically shows a longitudinal cross section view of a treatment solution generating device in which the passage body has a porous structure;

FIG. 6 diagrammatically shows a perspective view of a rolled porous membrane, e.g., for use in haemodialysis;

FIG. 7 diagrammatically shows a longitudinal cross section view of a treatment solution generator device in which the passage body is made of the rolled porous membrane of FIG. 5;

FIG. 8 diagrammatically shows a longitudinal cross section view of a treatment solution generating device in which the passage body has a granular structure and is formed from a loose mineral particulate material;

FIG. 9 is a diagram showing how the pressure of the ozone/ROS water solution changes along a path between the ozone water solution generator device and the nebulizer of FIG. 1;

FIG. 10 is a flow diagram of an apparatus according to an embodiment of the present invention, in which the mixer device comprises a reservoir;

FIG. 11 is a flow diagram of an apparatus according to a modification of the embodiment of FIG. 10, in which a hydrogen peroxide supply device is provided to supply H₂O₂ to the water-ozone mixer device;

FIG. 12 is a flow diagram of an apparatus according to an embodiment of the present invention, in which mixer device comprises a mixing duct enclosing a static mixer;

FIG. 13 is a flow diagram of an apparatus according to a modification of the embodiment of FIG. 11, in which a hydrogen peroxide supply device is provided to supply H₂O₂ to the water-ozone mixer device;

FIG. 14 is a flow diagram of an apparatus according to a modification of the embodiment of FIG. 10, wherein a compensation and/or recycle duct is provided between the treatment solution generator device according to one embodiment and the reservoir of the ozone water solution generator device;

FIG. 15 is a flow diagram of an apparatus for making a treatment solution containing reactive oxygen species and for stabilizing the reactive oxygen species by incorporation of the solution into a gel.

### DESCRIPTION OF SOME PREFERRED EMBODIMENTS

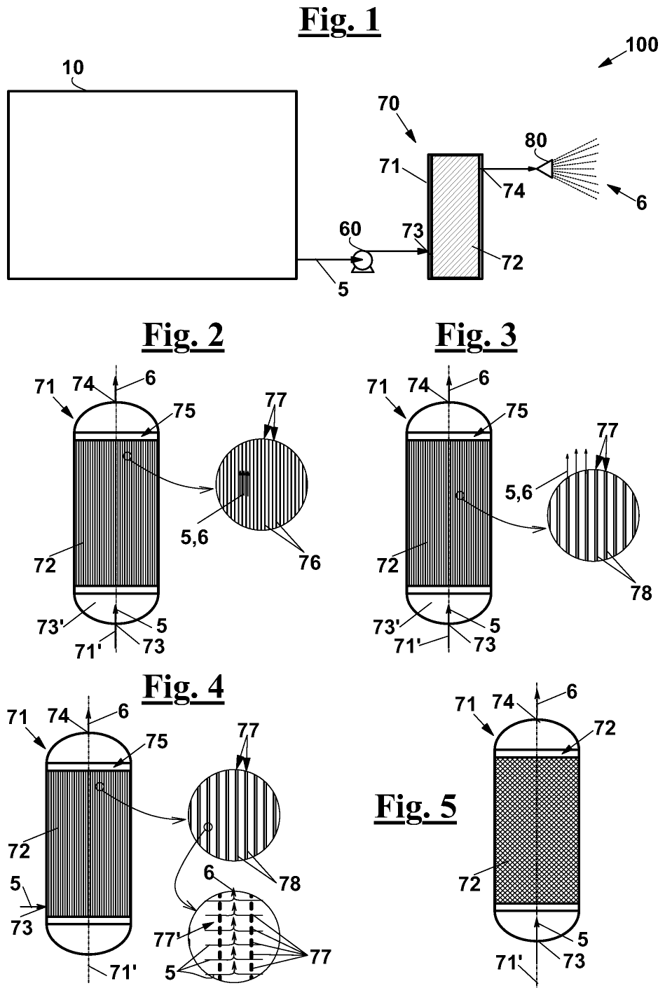

With reference to FIG. 1, an apparatus 100 is described for obtaining a treatment solution 6 containing reactive oxygen species (ROS), in particular superoxide anions O₂⁻.

Apparatus 100 comprises an ozone water solution generator device 10, described more in detail hereinafter with reference to FIGS. 2-8, and a treatment solution generator device 70 arranged in hydraulic connection with the ozone water solution generator device 10. Treatment solution generator device 70 is configured to turn an ozone water solution 5 obtained by ozone water solution generator device 10 into treatment solution 6 containing reactive oxygen species.

In the treatment solution generator device 70, a container 71 has a first opening 73 and a second opening 74, and encloses a passage body 72. First opening 73 is arranged in hydraulic connection with an outlet of ozone water solution generator device 10, so that ozone water solution 5 generated by ozone water solution generator device 10 can be supplied to first opening 73 of treatment solution generator device 70. As described in more detail below, a supply pump 60 can be provided to supply ozone water solution 5 to generating treatment solution generator device 70.

As shown in FIGS. 2-8, passage body 72 includes a plurality of passageways 77 having a predetermined passage-section size, in which ozone water solution 5 is turned into treatment solution 6 while flowing therethrough, i.e., in which ozone is converted into ROS as ozone water solution 5 advances within passage body 72.

The passage-section size of passageways 77 is set between 0.001 μm and 0.2 μm, preferably between 0.005 μm and 0.015 μm, in order to allow and possibly ozone conversion to ROS, in particular to O₂⁻ and O⁺ ions, as described hereinafter.

In particular, in the embodiments shown in FIGS. 2-8, container 71 has an elongated shape extending along a longitudinal axis 71′. Advantageously, but not exclusively, container 71 can have a cylindrical shape. In these embodiments, first opening 73, i.e., the inlet opening of ozone water solution 5 and second opening 74, i.e. the outlet opening of treatment solution 6 are located at opposite end portions of elongated container 71.

In the embodiment of FIG. 2, passage body 72 comprises a bundle 75 of full fibres 76 arranged in the direction of longitudinal axis 71′ of container 71. Full fibres 76 are packed together in such a way as to define, between one solid fibre 76 and the other, passageways 77 for ozone water solution 5 and for treatment solution 6 being formed. An empty space 73′ is advantageously provided between first opening 73 and fibre bundle 75 to obtain a uniform distribution of incoming ozone water solution 5 over the cross section of fibre bundle 75.

In the embodiment of FIG. 3, passage body 72 comprises a bundle 75 of hollow fibres 78 also arranged in the direction of longitudinal axis 71′. Hollow fibres 78 define within themselves respective passageways 77 for ozone water solution 5 and for treatment solution 6 being formed. In this case, the passage-section size corresponds to the internal cross section of hollow fibres 78. Also in this case, empty space 73′ is preferably provided between first opening 73 and fibre bundle 75 to promote uniform distribution of ozone water solution 5 in passage body 72. Hollow fibres 78 are tightly packed to maximise the number of passageways 77 of passage body 72. Preferably, fibre bundle 75 of hollow fibres 78 is mounted between two end plates, in the same manner as the tubes of a shell-and-tube heat exchanger.

FIG. 4 shows a modification of the embodiment of FIG. 3, in which the wall of hollow fibres 78 has submicrometric porosities 77 that place the inner lumen of hollow fibres 78 into hydraulic communication with the outside of hollow fibres 78 themselves, within container 71, similar to the shell-side of a shell-and-tube heat exchanger. In this case, passageways 77, through which water solution 5,6 containing ozone and the ROS being formed flows, can include or be the porosities 77 of the wall of hollow fibres 78.

In this arrangement, first and second openings 73,74 are preferably arranged such that ozone water solution 5 turning into treatment solution 6 flows from shell side 79 into the tube-side of bundle 75 through porosities 77, i.e, first opening 73 is made through a wall of the shell portion of container 71, whereas second opening 74 is made through the wall of the bonnet portion of container 71.

In particular, hollow fibres 78 of passage body 72 can be of the same type used in a dialyzer.

In particular hollow fibres 78 of passage body 72 can be of the same type used to perform an ultrafiltration of a liquid flowing therethrough, i.e., through the porosities of the wall of hollow fibres 78, typically from outside to inside hollow fibres 78.

Passage bodies 72 shown in FIGS. 2 to 4, i.e., full fibres 76 and hollow fibres 78, can be made of various materials. In particular, polysulfone, cellulose triacetate and polyvinyl chloride are preferred, as well as polypropylene, polyethersulfone, well known to a person skilled in the art of ultrafiltration.

In the embodiment of FIG. 5, passage body 72 is a porous compact body, not including a tube or tube bundle structure. In this case, passageways 77 for ozone water solution 5 being turned into ROS-containing treatment solution 6 are defined by a percolable open-cell lattice, and the passage-section size corresponds to the cell size of the lattice. For instance, porous compact passage body 72 can be obtained in a known manner by sintering metal or ceramic powders. In a modification of this embodiment, passage body 72 with porous structure may be an open-cell sponge.

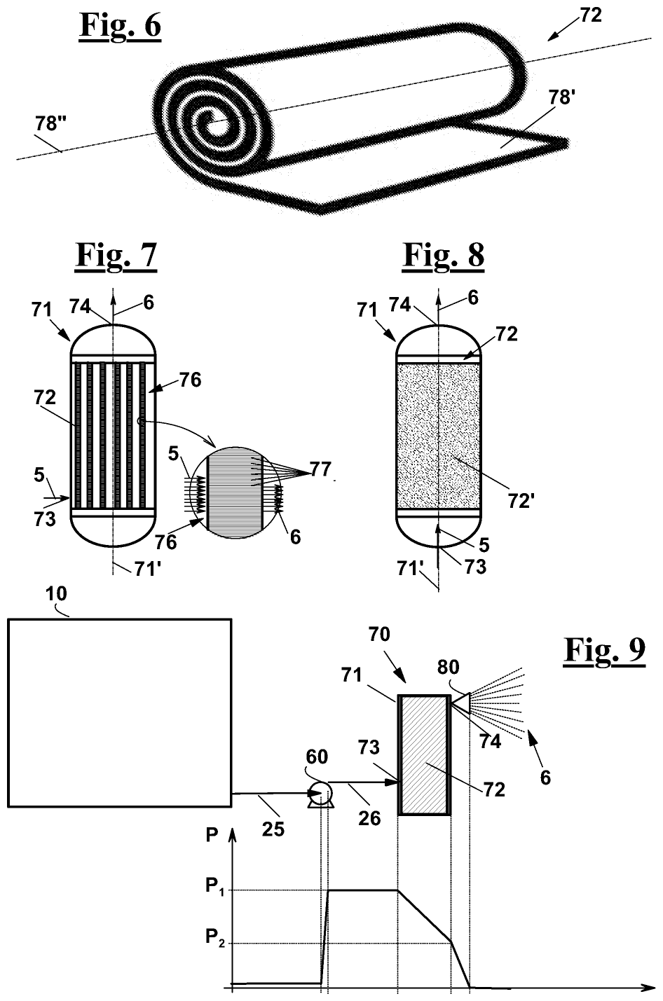

FIGS. 6 and 7 relates to an embodiment in which passage body 72 is a porous body consisting of a rolled porous membrane 78′, folded about an axis 78″ thereby forming a substantially cylindric structure suitable for insertion into container 71 having a cylinder shape.

Also in this case, in particular, porous membrane 78′ of passage body 72 can be of the same type as used in a dialyzer.

Also in this case, in particular porous membrane 78′ of passage body 72 can be of the same type as used to perform an ultrafiltration of a liquid flowing therethrough, i.e., through porosities 77 of membrane 78′, typically towards axis 78″.

Passage bodies 72 shown in FIGS. 6 and 7, i.e., porous membrane 78′ can be made of various materials. In particular, polysulphone, cellulose triacetate, polylactic acid and polyvinyl chloride are preferred, as well known to a person skilled in the art of ultrafiltration.

In the embodiments of FIG. 8, passage body 72 is formed of a loose mineral particulate material 72′ whose grain size is selected in such a way to form also in this case a percolable open-cell lattice in which the cells have a predetermined size, as provided by the method. For instance, loose mineral particulate material 72′ can be a sand of a controlled grain size, i.e. a screened sand appropriately to obtain the desired passage-section size. As an alternative, the loose mineral particulate material may be a mineral known in the technique as an ultrafiltration aid.

A plurality of preferably serially arranged passage bodies 72 can be provided along the flowpath of ozone water solution 5 of the types described above.

Advantageously, before or after passing through passageways 77, ozone water solution 5, or ROS-containing treatment solution 6, respectively, can flow through duct portions 77′ (FIG. 4) that are provided in the passage body 72. Duct portions 77′ can have a section size set between 10 μm and 1 mm, preferably between 50 μm and 500 μm, more preferably between 100 μm and 300 μm.

Downstream of treatment solution generator device 70, in hydraulic connection with second opening 74, there is provided a nebulizer 80, preferably configured to split a liquid flow available at a predetermined second pressure P₂ into submillimetric particles, more preferably micron-sized particles, and arranged then to release treatment solution 6 outside of the generating device 70, in such a particle form.

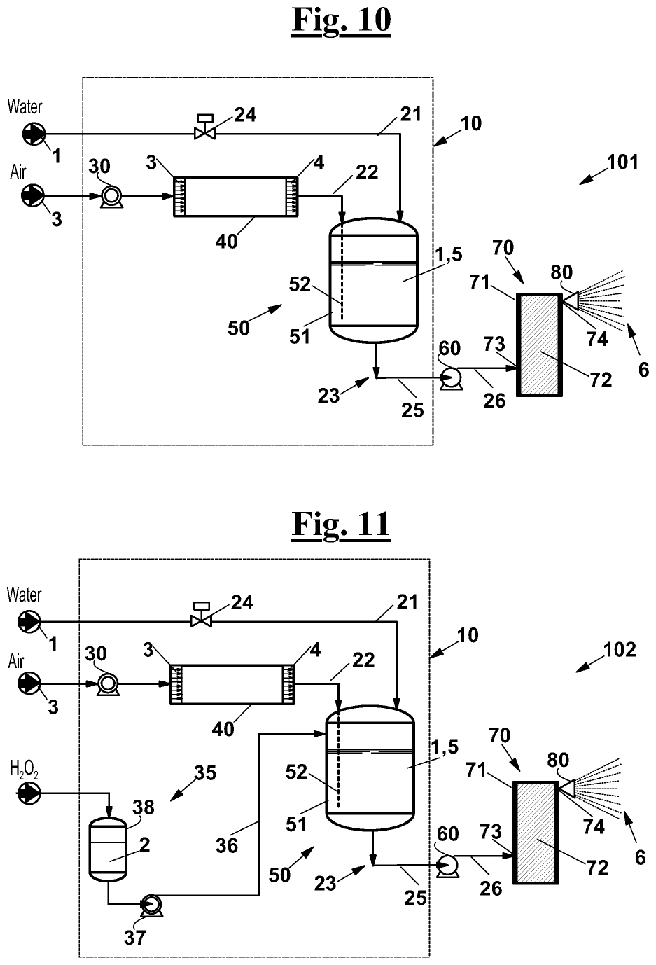

Supply pump 60 is selected in such a way that ozone water solution 5 reaches container 71 at a predetermined first pressure P₁, preferably set between 7 and 15 bar g, in particular between 10 and 12 bar g. The water solution pressure profile along apparatus 100 is schematically shown in FIG. 9.

Moreover, passage body 72 is arranged to release treatment solution 6 to nebulizer device 80 at second pressure P₂ set between 4 bar g and 7 bar g.

This way, ozone water solution 5 can flow through passageways 77 of passage body 72, for example the inner lumen of hollow fibres 78 of FIG. 3 or the porosities 77 of the wall of hollow fibres 78 of FIG. 3, or the percolable lattice of a porous or granular body 72, as in FIGS. 5 to 8. During this passage, the combined effect of the pressure and the interactions with the inner walls of passageways 77 enhances the instability of the ozone molecules O₃ contained in ozone water solution 5, whereby the ozone is at least in part turned into ROS, e.g., an ozone heterolytic cleavage reaction takes place forming anions O₂⁻ and cations O⁺:

2O₃→O₂⁻+O⁺.

Second opening 74 then produces treatment solution 6 containing reactive oxygen species.

Treatment solution 6 generated in passage body 72 then flows through nebulizer 80 and is available for sprinkling a biological system, or for further ROS stabilization, as described hereinafter.

In the first case, reactive oxygen species from the treatment solution can be deposited directly onto the biological system, with which they can interact before spontaneously decaying to molecular oxygen O₂.

With reference to FIG. 10, there is described an apparatus 101, according to an embodiment of the invention, for producing and administering a treatment solution 6 containing reactive oxygen species, in particular O₂⁻ anions, from water 1 and an oxygen-containing gas 3, in particular air. For the sake of brevity, reference will be made in the following description to air 3 taken from the environment as an oxygen-containing gas. However, in embodiments not shown in the drawings, the oxygen-containing gas may be a gas distinct from atmospheric air, for example substantially pure oxygen, or compressed air taken from a portable pressure vessel.

Apparatus 101 is configured to at least in part transform the oxygen contained in air 3 into ozone, from which the superoxide anions of treatment solution 6 are then obtained, as described below.

Apparatus 101 comprises a conventional ozone generator 40 configured to convert at least one portion of the oxygen contained in air 3 into ozone O₃. Associated with the ozone generator 40 there is a fan 30 arranged to convey a air stream 3 taken from the environment, at a predetermined flow rate, through the generator 40. In the embodiment of FIG. 10, fan 30 is arranged upstream of the ozone generator 40, but in other embodiments it may be arranged downstream of it.

The ozone generator 40 thus produces a gas 4 containing ozone in addition to nitrogen and any unconverted oxygen, and smaller quantities of other gases normally contained in the air.

Apparatus 101 further comprises a mixer device 50 configured to bring an amount or a stream of water 1 into contact with ozone-containing gas 4, so as to dissolve the ozone into water 1 and obtain ozone water solution 5.

In the embodiment of FIG. 10, mixer device 50 comprises a reservoir 51 for receiving a predetermined amount of water 1. For this purpose, mixer device 50 is associated with a water supply means 21, for example, as shown in FIG. 10, a supply line 21 from a water supply network. In a modification, not shown, water supply means 21 can comprise a hopper arranged to receive water 1 and to selectively put it into communication with reservoir 51, so as to transfer water 1 into reservoir 51 by gravity.

Apparatus 101 further comprises a supply line 22 of ozone-containing gas stream 4, along which fan 30 and the ozone generator 40 are arranged as described above. Supply line 22 and reservoir 51 are preferably arranged to supply ozone-containing gas stream 4 below the level of the liquid 1,5 contained in reservoir 51, corresponding to the amount of water 1, so as to bring ozone-containing gas 4 into contact with water 1. In the embodiment shown in FIG. 10, reservoir 51 is provided with a submerged tube 52 in hydraulic connection with supply line 22 of ozone-containing gas 4. In some modifications of the present embodiment, not shown mixer device 50 comprises a conventional gas-to-liquid diffuser means for finely dispersing ozone-containing gas 4 into water 1 contained in reservoir 51. Such diffuser means can be arranged at the submerged end, i.e., at the outlet of submerged tube 52.

Apparatus 101 further comprises a discharge duct 23 of ozone water solution 5, along which pump 60 is arranged, thus defining a suction portion 25 and a delivery portion 26 of discharge duct 23. Delivery portion 26 of discharge duct 23 is connected to treatment solution generator device 70.

FIG. 11 shows an apparatus 102, according to an embodiment of the invention, which differs from apparatus 101 of FIG. 10 in that it comprises a hydrogen peroxide supply device 35 for supplying hydrogen peroxide H₂O₂ 2 to reservoir 51 of mixer device 50. Hydrogen peroxide supply device 35 may comprise a feed line 36 and a pump 37, such as a metering pump, arranged to transfer a predetermined amount of hydrogen peroxide 2 from an hydrogen peroxide container 38. Hydrogen peroxide container 38 can be a container of hydrogen peroxide as purchased from a supplier, or a fixed tank 38 of apparatus 102.

Supply means or devices 21, 22, 35 of water 1, ozone-containing gas 4 and hydrogen peroxide 2 can be equipped with respective mass or flow rate predetermination means fed to supply predetermined amounts of water, ozone and hydrogen peroxide to mixer device 50, in particular to reservoir 51. In the case of water supply lines 1 and hydrogen peroxide 2 as feed means, such predetermination means may be flow meters configured to emit electrical signals upon reaching a predetermined amount of the liquid to be fed to mixer device 50, in order to close a shut-off valve 24 of the water supply line 21 of water 1 or to stop the hydrogen peroxide supply pump 37. In the case of loading hoppers as feed means, the predetermination means may comprise weighing devices or level indicators. The aforementioned predetermination means are of a conventional type and therefore easily implemented by a technician in the branch, hence they are not described in detail, nor are they shown in the drawings.

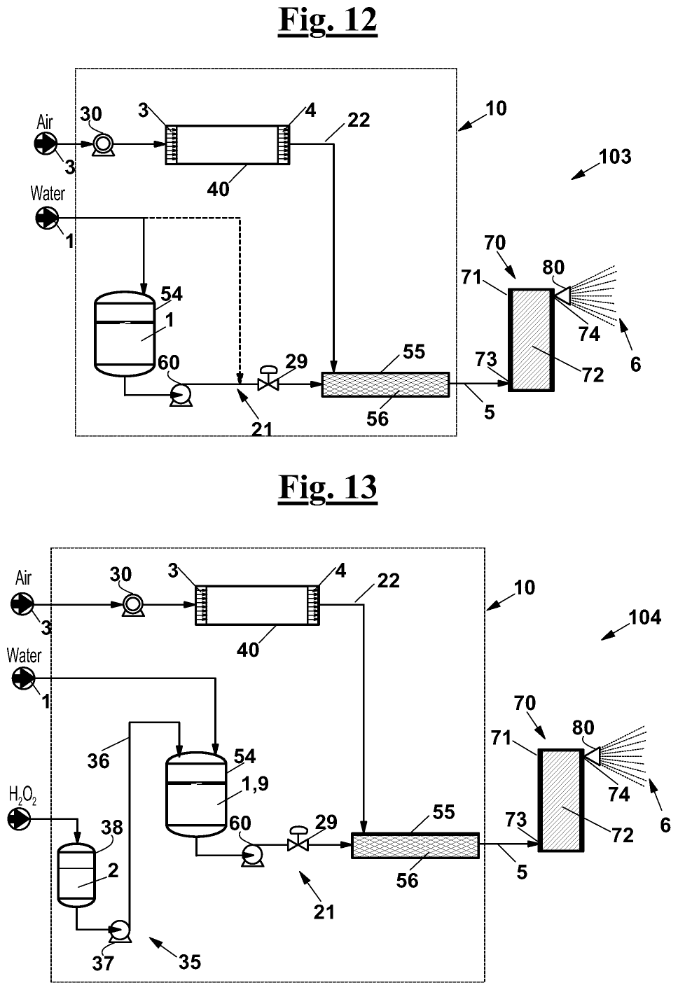

FIG. 12 relates to an apparatus 103 according to a further embodiment of the invention, which differs from apparatus 101 of FIG. 10 in that mixer device 50 comprises, instead of the mixing tank 51, a mixing duct 55, in this case a tubular element within which a conventional static mixer 56 is arranged.

Again, apparatus 103 comprises a water supply means 21 and an ozone-containing gas supply device 22. Water supply means 21 may comprise a feed tank 54, a supply pump 60 and, preferably, a water flow rate control valve 29 for setting the correct flow rate to mixing duct 55. Pump 60 and the regulating valve 29 are selected in such a way to supply water 1 to mixing duct 55 at the pressure required by static mixer 56. As an alternative, shown by a dotted, water 1 can be directly withdrawn from a distribution network in which water 1 is available at a pressure at least equal to the pressure required by static mixer 56, without requiring feed tank 54 and pump 60 to convey water 1 to mixing duct 55.

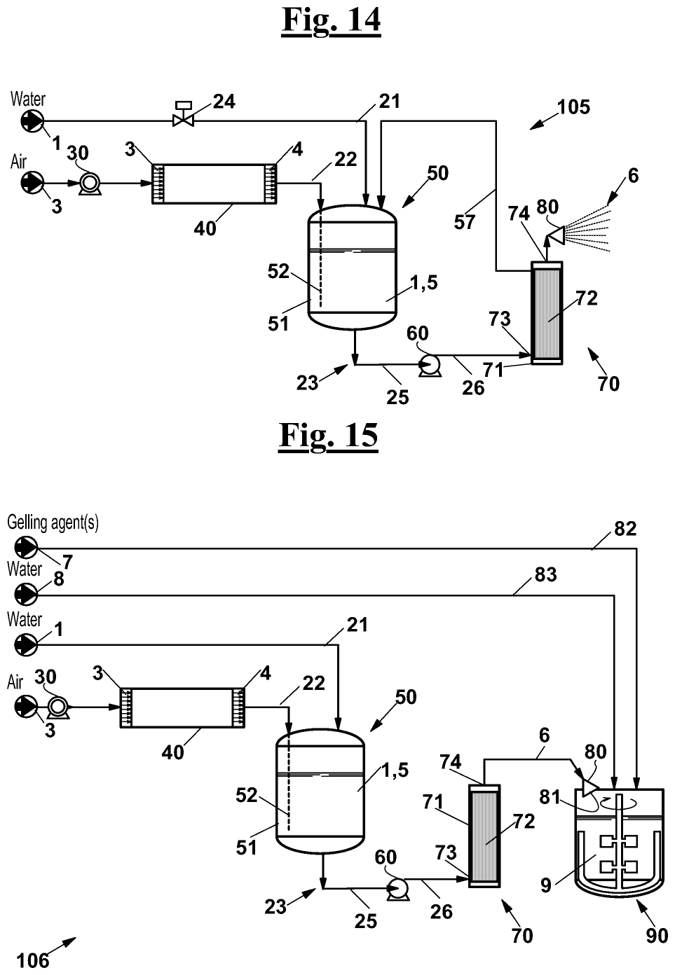

FIG. 13 shows an apparatus 104 according to an embodiment of the invention, which differs from apparatus 103 of FIG. 12 in that it comprises hydrogen peroxide supply means 35 for supplying hydrogen peroxide 2 to the mixer tank 54 similarly to apparatus 102 of FIG. 11. In this case, the correct hydrogen peroxide/water proportion is advantageously set in feed tank 54.

Referring again to FIGS. 9-12, container 71 of treatment solution generator device 70 has first opening 73 hydraulically connected to outlet duct 23 of ozone water solution 5, while second opening 74 of the superoxide anion generator 70 is preferably directly connected to nebulizer 80.

FIG. 14 shows an apparatus 105 which differs from apparatus 101 of FIG. 10 in that container 71 of treatment solution generator device 70, in particular, a space 79 defined between the shell of container 71 and hollow fibres 78, is hydraulically connected with an opening of reservoir 51 via a compensation and recycling duct 57, in order to maintain the pressure within container below a predetermined safety value. As an alternative to the embodiment of FIG. 14, an appropriately set safety valve can be provided on one wall of container.

FIG. 15 diagrammatically shows an apparatus 106 according to a further embodiment of the invention, comprising such a gel-forming turbomixer 90 hydraulically connected to an output mouth 81 of nebulizer device 80 so as to be supplied by treatment solution 6 as released by nebulizer device 80. Moreover, gel-forming turbomixer 90 can be associated to a gelling agent 7 supply means 82 and to a water 8 further supply means 83 so as to prepare gel 9 within gel-forming turbomixer 90, or to compensate for the viscosity change of a preformed gel 9 due to the addition of the water accompanying the ROS in treatment solution 6.

### EXAMPLES

Production tests of ROS-containing treatment solution 6 were conducted using a prototype apparatus according to the diagram of of apparatus 105 in FIG. 14, in which:
fan 30 is a fan capable of delivering a 0.3 mc/hr air flow rate of at a 2.4 mbar delivery pressure;
ozone generator 40 is an 80 W generator;
tank 51 of mixer device 50 has a capacity of 30 litres;
supply pump 60 is a rotary pump capable of delivering a 20 litres/minute water flow rate at an 1.8 bar delivery pressure;
treatment solution generating device 70 comprises a Ø3.2×27 mm container as shown FIG. 4, in which bundle 75 of 200 μm diameter hollow polysulphone fibres, more in detail, a Fresenius mod. F5HPs type dialyser was used;
nebulizer 80 is a brass atomiser of one type normally used for irrigation.

Tests were conducted under the following operating conditions:
ozone water solution throughput: 30 litres;
duration of the ozone water solution generation step (uptime of ozone generator 40): approx. 1 minute;
feed rate to treatment solution generator 70:20 litres/minute;

In the tests in Examples 2 and 4, an amount of 35% hydrogen peroxide 2 was added to water 1 to prepare ozone water solution 5, set to 0.2 ml/l of of water in reservoir 51;

An electrical conductivity measurement was performed on the freshly generated treatment solution 6 by a Hanna EDGE instrument capable of converting the measurement to ion concentration values, provided a preliminary calibration is performed to exclude the contribution of ions that are normally present in water.

The results are shown in the table below, along with a comparison example of a treatment solution obtained with a prior art device described in IT 102018000009939.

| Example | Water | H₂O₂ in water | ROS concentration |
|---|---|---|---|
| comparison | demineralised | — | 10-20 |
| n. 1 | demineralised | No | 370 |
| n. 2 | demineralised | yes | 402 |
| n. 3 | potable | no | 460 |
| n. 4 | potable | yes | 688 |

The above results show a significant reactive oxygen species concentration increase in comparison to what is possible with the prior art device; a significant effect of hydrogen peroxide, which is particularly important in combination with the salts dissolved in the water; and a significant effect of the dissolved salts themselves.

Tests carried out at various concentrations of the salts dissolved in the water, and with different amounts of hydrogen peroxide, confirmed an increasing effect of these factors, in terms of the concentration of ROS ion in the treatment solutions obtained, starting from values as low as those indicated in the first line of the table, up to values as high as those indicated in the other lines, beyond which a saturation effect tends to occur.

The above description of certain specific embodiments, as well as the examples provided, are capable of showing the invention from a conceptual point of view in such a way that others, using the known technique, will be able to modify and/or adapt that specific embodiment in various applications without further research and without departing from the inventive concept, and, therefore, it is understood that such adaptations and modifications will be considered as equivalents of the specific embodiment. The means and materials for realising the various functions described may be of various kinds without departing from the scope of the invention. It is understood that the expressions or terminology used are purely descriptive and, therefore, not limiting.

1. A method for obtaining a treatment solution containing reactive oxygen species capable of treating a biological system, said method comprising:
prearranging a container with a first opening and a second opening, and a passage body within said container, between said first opening and said second opening;
wherein said passage body includes a plurality of passageways having a predetermined passage-section size;
prearranging a nebulizer device in hydraulic connection with said second opening;
generating an ozone water solution;
feeding said ozone water solution into said first opening of said container at a predetermined first pressure, such that
said ozone water solution is caused to flow through said plurality of passageways of said passage body, wherein said predetermined passage-section size is selected to convert said ozone of said ozone water solution into reactive oxygen species, generating a treatment solution at a second pressure lower than said first pressure;
said treatment solution is immediately thereafter caused to flow through said nebulizer device and is then nebulized into submillimetric particles which can be;
directly deposited on said biological system and said ROS can interact with said biological system without decaying into molecular oxygen O₂ or which can be preserved;
wherein said passage body is configured to cause said pressure to drop from said first pressure of said ozone water solution to said second pressure of said treatment solution passing therethrough, and is selected from the group consisting of:
a bundle of hollow fibers, and said passageways for said ozone water solution and for said treatment solution are defined within said hollow fibers;
a bundle of full fibers, and said passageways include empty spaces among said full fibers;
a porous body, wherein said passageways are defined by a percolable open-cell lattice having a predetermined cell size;
a body comprising a loose mineral particulate material; and
a combination of the above-mentioned passage bodies.

2. (canceled)

3. The method according to claim 1, wherein said loose mineral particulate material is a sand or an ultrafiltration aid, said loose mineral particulate material having a grain size selected in such a way to form a percolable open-cell lattice having a predetermined cell size.

4. (canceled)

5. The method according to claim 1, wherein said passage-section size of said passageways in said passage body is selected between 0.001 μm and 0.2 μm.

6. The method according to claim 1, wherein said step of generating an ozone water solution comprises the steps of:
generating ozone from oxygen, obtaining an ozone-containing gas stream;
contacting said ozone-containing gas stream with water in a mixer device and dissolving said ozone into said water, so as to form said ozone O₃ water solution;
wherein said water includes a predetermined amount of hydrogen peroxide, and said step of contacting and dissolving ozone into water takes place in the presence of said hydrogen peroxide.

7. The method according to claim 6, wherein said amount of hydrogen peroxide is equivalent to a 35% hydrogen peroxide water solution amount set between 1/500 and 1/5000 of an amount of said water.

8. The method according to claim 1, wherein said water contains ions selected from the group consisting of: Na⁺, K⁺, Ca²⁺, Mg²⁺, Fe²⁺, Cl⁻, SO₄⁻, HCO₃⁻, F⁻, and NO₃⁻, each present at a concentration set between 50 and 500 mg/liter.

9. A method for stabilizing reactive oxygen species including superoxide anion O₂⁻ of a treatment solution manufactured according to claim 1, said method further comprising incorporating said submillimetric particles of said treatment solution into a gel.

10. The method according to claim 9, wherein incorporating said submillimetric particles into a gel comprises:
prearranging an amount of said gel in a gel-forming turbomixer;
maintaining said gel under agitation in said gel-forming turbomixer;
supplying said treatment solution as released by said nebulizer device into said gel-forming turbomixer during said step of maintaining said gel under agitation.

11. The method according to claim 10, wherein prearranging an amount of said gel comprises:
prearranging an amount of a gelling agent in said gel-forming turbomixer;
supplying a predetermined amount of water to said gel-forming turbomixer; and
preliminary maintaining said gelling agent and said water under agitation in said gel-forming turbomixer.

12. An apparatus for making a treatment solution containing reactive oxygen species, and for treating a biological system with said treatment solution, said apparatus comprising:
an ozone water solution generator device;
a treatment solution generator device,
wherein said treatment solution generator device is arranged in hydraulic connection with said ozone water solution generator device,
wherein said treatment solution generator device comprises:
a container with a first opening and a second opening, and
a passage body arranged within said container between said first opening and said second opening,
wherein said passage body includes a plurality of passageways having a predetermined passage-section size;
a nebulizer device in hydraulic connection with said second opening of said container, said nebulizer device arranged to release said treatment solution outside of said generating device in the form of submillimetric particles;
a supply pump arranged to supply said ozone water solution into said first opening of said container at a predetermined first pressure, such that
said ozone water solution flows through said passage body, forming said treatment solution at a second pressure lower than said first pressure;
said treatment solution at said second pressure subsequently flows through said nebulizer device and is released outside of said treatment solution generator device;
wherein said plurality of passageways of said passage body, present said predetermined passage-section size selected to convert said ozone of said ozone water solution into said treatment solution, such that reactive oxygen species are dissolved in said treatment solution and can be directly sprinkled by said nebulizer device without decaying into molecular oxygen O₂;
wherein said passage body is configured to cause said pressure to drop from said first pressure of said ozone water solution to said second pressure of said treatment solution passing therethrough, and is selected from the group consisting of:
a bundle of hollow fibers, and said passageways for said ozone water solution and for said treatment solution are defined within said hollow fibers;
a bundle of full fibers, and said passageways are defined by empty spaces among said full fibers;
a porous body, wherein said passageways are defined by a percolable open-cell lattice having a predetermined cell size; and
a combination of the above-mentioned passage bodies.

13. The apparatus according to claim 12, wherein said hollow fibers are made of a material selected from the group consisting of: a polysulphone, cellulose triacetate and polyvinyl chloride.

14. The apparatus according to claim 12, wherein said porous body is selected from the group consisting of:
a ceramic or metal sintered body; and
an open-cell sponge.

15. The apparatus according to claim 12, wherein said loose mineral particulate material is a sand or an ultrafiltration aid, said loose mineral particulate material having a grain size selected such as to form a percolable open-cell lattice having a predetermined cell size.

16. (canceled)

17. The apparatus according to claim 12, wherein said passage-section size of said passageways in said passage body is selected between 0.001 μm and 0.2 μm.

18. The apparatus according to claim 12, wherein said supply pump is arranged to supply said ozone water solution into said first opening of said container at said predetermined first pressure set between 7 bar g and 15 bar g.

19. The apparatus according to claim 18, wherein said passage body is arranged to release said treatment solution to said nebulizer device at said second pressure set between 4 bar g and 7 bar g.

20. The apparatus according to claim 12, wherein said container has an elongated shape and said first opening and said second opening serving as an inlet opening of said ozone water solution and as an outlet opening of said treatment solution, respectively, are arranged at opposite end portions of said container.

21. The apparatus according to claim 12, wherein said ozone water solution device generator comprises:
a water and ozone mixer device;
an ozone generator pneumatically connected to said mixer device and arranged to receive an atmospheric air stream and to change said atmospheric air stream into an ozone-containing gas stream;
an ozone supply unit for supplying said ozone into said mixer device, said ozone supply unit arranged to bring said ozone-containing gas stream into contact with said water contained in said mixer device, so that said ozone dissolves into said water forming said ozone water solution O₃;
wherein said water and ozone mixer device is selected from the group consisting of:
a mixer device comprising a reservoir configured to contain a predetermined amount of water, and said ozone supply unit and said reservoir are arranged to supply said ozone-containing gas stream below a level corresponding to said amount of water in said reservoir; and
a mixer comprising a mixing duct within which a static mixer is arranged.

22. The apparatus according to claim 12, further comprising a gel-forming turbomixer configured for containing an amount of a gel and for maintaining said gel under agitation,
wherein said gel-forming turbomixer is hydraulically connected to an output mouth of said nebulizer device so as to be supplied by said treatment solution as released by said nebulizer device.

23. The apparatus according to claim 12, wherein said gel-forming turbomixer is associated with:
a gelling agent supply means;
a water supply means;
so as to prepare said gel within said gel-forming turbomixer.
